(19) United States
(12) Patent Application Publication
(10) Pub. No.: US 2009/0259221 A1
(43) Pub. Date: Oct. 15, 2009

(54) POWER SUPPLY APPARATUS FOR OPERATION

(76) Inventors: Naoko Tahara, Hachioji-shi (JP); Koh Shimizu, Kodaira-shi (JP)

Correspondence Address: OSTROLENK FABER GERB & SOFFEN, 1180 AVENUE OF THE AMERICAS, NEW YORK, NY, 100368403, US

(21) Appl. No.: 12/102,994

(22) Filed: Apr. 15, 2008

(51) Int. Cl. A61B 18/14

(52) U.S. Cl. 606/34

(57) ABSTRACT

A power supply apparatus for operation for outputting power to a surgical instrument includes an impedance detection section for detecting the impedance of the surgical instrument in the output, and an abnormality detection section for detecting an abnormality according to whether or not a variation value of the impedance per unit time exceeds a predetermined first impedance variation value. The abnormality detection section further detects an abnormality according to whether or not a variation value of a resonant frequency per unit time exceeds a predetermined threshold. The abnormality is detected in this manner, whereby it is possible to prevent the surgical instrument from being broken.

### BACKGROUND OF THE INVENTION

1. Field of the Invention

The present invention relates to a power supply apparatus for operation.

2. Description of the Related Art

A drive apparatus for an ultrasonic vibrator is hitherto known as a power supply apparatus for operation. For example, in Jpn. Pat. Appln. KOKAI Publication No. 7-303635, it is disclosed that in a vibrator drive circuit employing phase-locked loop (PLL) control, means for switching PLL transient characteristics is provided, and stability is obtained in a step of thereafter performing a resonance point tracking operation. Further, in Jpn. Pat. Appln. KOKAI Publication No. 2003-159259, a method for discriminating between damage of a defective hand-piece and damage of a defective blade in an ultrasonic surgical system is disclosed. Further, in US2002-0049551, a method for clarifying the difference between a loaded blade and a cracked blade is disclosed.

### BRIEF SUMMARY OF THE INVENTION

A first aspect of the present invention relates to a power supply apparatus for operation for outputting power to a surgical instrument, the apparatus comprising: an impedance detection section for detecting the impedance of the surgical instrument in the output; and an abnormality detection section for detecting whether or not a variation value of the impedance per unit time exceeds a predetermined first impedance variation value.

Further, a second aspect of the present invention relates to the first aspect, and the abnormality detection section further detects whether or not a variation value of a resonant frequency per unit time exceeds a predetermined threshold.

Further, a third aspect of the present invention relates to a power supply apparatus for operation for outputting power to a surgical instrument, the apparatus comprising: a detection section for detecting an output voltage or an output current in the output; and an abnormality detection section for detecting whether or not a variation value of the output voltage or the output current per unit time exceeds a predetermined first voltage variation value or a predetermined first current variation value.

Further, a fourth aspect of the present invention relates to the first aspect, and each of intervals at which the impedance is detected is 10 msec or less.

Further, a fifth aspect of the present invention relates to the first aspect, and the first impedance variation value is 600Ω/100 msec or more.

Further, a sixth aspect of the present invention relates to the first aspect, and the abnormality detection section stops outputting the power to the surgical instrument when the variation value of the impedance per unit time exceeds the first impedance variation value.

Further, a seventh aspect of the present invention relates to the third aspect, and the abnormality detection section stops outputting the power to the surgical instrument when the variation value of the output voltage or the output current exceeds the predetermined first voltage variation value or the predetermined first current variation value.

Further, an eighth aspect of the present invention relates to the first or third aspect, and the surgical instrument is provided with an ultrasonic vibrator, and a probe for transmitting the vibration of the ultrasonic vibrator to a distal end thereof, and the output power is ultrasonic power for driving the ultrasonic vibrator.

Further, a ninth aspect of the present invention relates to the first aspect, and the abnormality detection section further detects whether or not the variation value of the impedance per unit time exceeds a second impedance variation value when a value of the impedance detected by the impedance detection section exceeds a predetermined reference value.

Furthermore, a tenth aspect of the present invention relates to the ninth aspect, and the second impedance variation value is smaller than the first impedance variation value.

Moreover, an eleventh aspect of the present invention relates to the tenth aspect, and the abnormality detection section stops supplying the power to the surgical instrument when the variation value of the impedance per unit time exceeds the first variation value, or when the value of the impedance exceeds the reference value, and the variation value of the impedance per unit time exceeds the second impedance variation value.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is an external perspective view of an ultrasonic operation system.

FIG. 2 is a view showing a schematic configuration of the ultrasonic operation system.

FIG. 3 is a view showing a state where a drive current generated in an ultrasonic power source unit flows to the hand-piece side.

FIG. 4 is a view showing a relationship between a voltage phase and a current phase.

FIG. 5 is a view for explaining a procedure for scanning for a resonant frequency fr.

(A) in FIG. 6 is a view showing a probe part in an enlarging manner.

(B) to (E) in FIG. 6 are graphs showing frequency dependence of impedance Z and a phase difference (θV−θI) which are under the PLL control, from a state where a probe is normal, through a state where the probe is cracked, to a state where the probe is broken.

FIG. 7 is a functional block diagram for explaining a function of each unit in the ultrasonic power source unit in the ultrasonic operation system.

FIG. 8 is a graph showing time dependence of impedance.

FIG. 9 is a flowchart for detecting an abnormality of a probe according to a first embodiment.

FIG. 10 is a flowchart for detecting an abnormality of a probe according to a second embodiment.

FIG. 11 is a flowchart for detecting an abnormality of a probe according to a third embodiment.

FIG. 12 is a flowchart for detecting an abnormality of another probe according to the third embodiment.

FIG. 13 is a functional block diagram for explaining a function of each unit in the ultrasonic power source unit in the ultrasonic operation system.

FIG. 14 is a graph showing time dependence of the frequency and impedance.

FIG. 15 is a graph showing time dependence of the frequency and impedance.

FIG. 16 is a flowchart for detecting an abnormality of a probe according to a sixth embodiment.

FIG. 17 is a flowchart for detecting an abnormality of another probe according to the sixth embodiment.

### DETAILED DESCRIPTION OF THE INVENTION

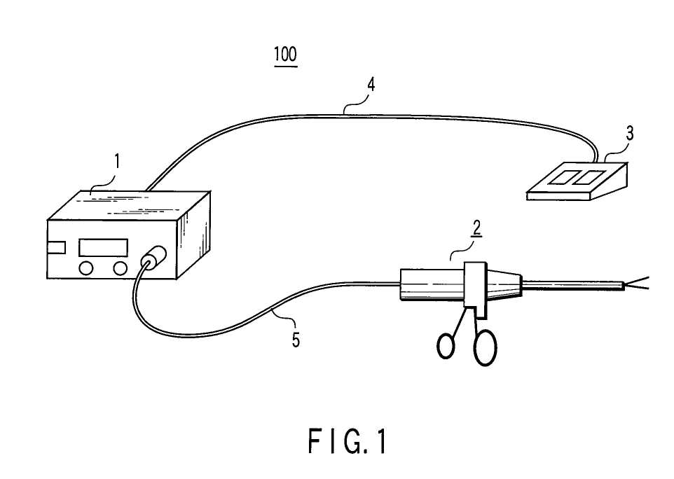

Embodiments of the present invention will be described below in detail with reference to the accompanying drawings. An endoscopic surgical operation for performing medical treatment of a diseased part to be performed by using a scope for observing a state in an abdominal cavity of a patient is known. FIG. 1 is an external perspective view of an ultrasonic operation system used as an example of a system for such an endoscopic surgical operation. The ultrasonic operation system is constituted of an ultrasonic power source unit 1 serving as a power supply apparatus for operation for generating an ultrasonic output for driving an ultrasonic vibrator, a hand-piece 2 serving as an ultrasonic surgical instrument for performing treatment by using an ultrasonic output supplied from the ultrasonic power source unit 1 through a cable 5, and a foot switch 3 connected to the ultrasonic power source unit 1 through a cable 4, for controlling the ultrasonic output from the ultrasonic power source unit 1.

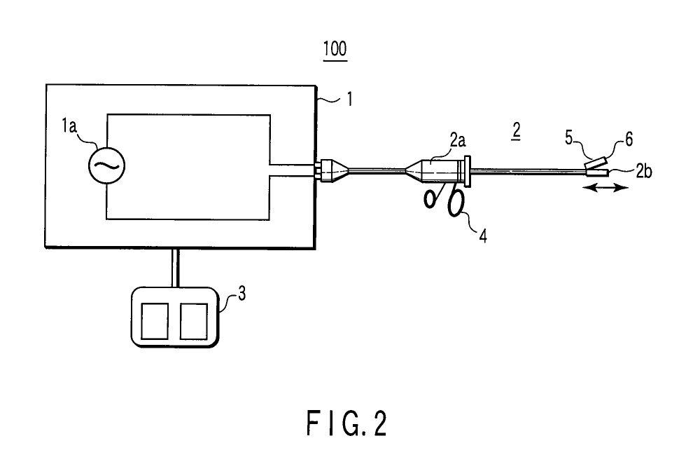

In FIG. 2, the hand-piece 2 is constituted of a hand-piece main body section 2a which includes handles 4, and in which an ultrasonic vibrator (not shown) is incorporated, and a probe 2b for transmitting vibration of the ultrasonic vibrator to a treatment section 5. The ultrasonic power source unit 1 is provided with an ultrasonic oscillator circuit 1a for generating electric energy for vibrating the ultrasonic vibrator. An electric signal output from the ultrasonic power source unit 1 is converted into mechanical vibration (ultrasonic vibration) by the ultrasonic vibrator inside the hand-piece main body section 2a, and thereafter the vibration is transmitted by the probe 2b to the treatment section 5. The treatment section 5 is provided with a grasping section 6 called a jaw driven to be opened or closed with respect to the distal end of the probe 2b. When the handles 4 are operated, the grasping section 6 is driven to be opened or closed with respect to the distal end of the probe 2b, and coagulation or incision of living tissue is performed by utilizing frictional heat generated by holding the living tissue between the distal end of the probe 2b and the grasping section 6 and applying the ultrasonic vibration thereto.

In this probe 2b, a crack is caused due to a scratch received when the probe 2b comes into contact with forceps or a clip during an operation. When a crack is caused to the probe 2b during an operation, it is necessary to immediately stop ultrasonic vibration, and replace the probe with a new one. If the operation is continued in the state where the crack is caused to the probe, it is conceivable that there is the possibility of the probe part being broken and falling off. Accordingly, it becomes necessary to detect the occurrence of the crack at an early stage, and inform the medical pursuer of the occurrence of the crack. The ultrasonic operation system will be described below in detail, and an apparatus and a method for exactly detecting an occurrence of a crack in a probe in an early stage will be described.

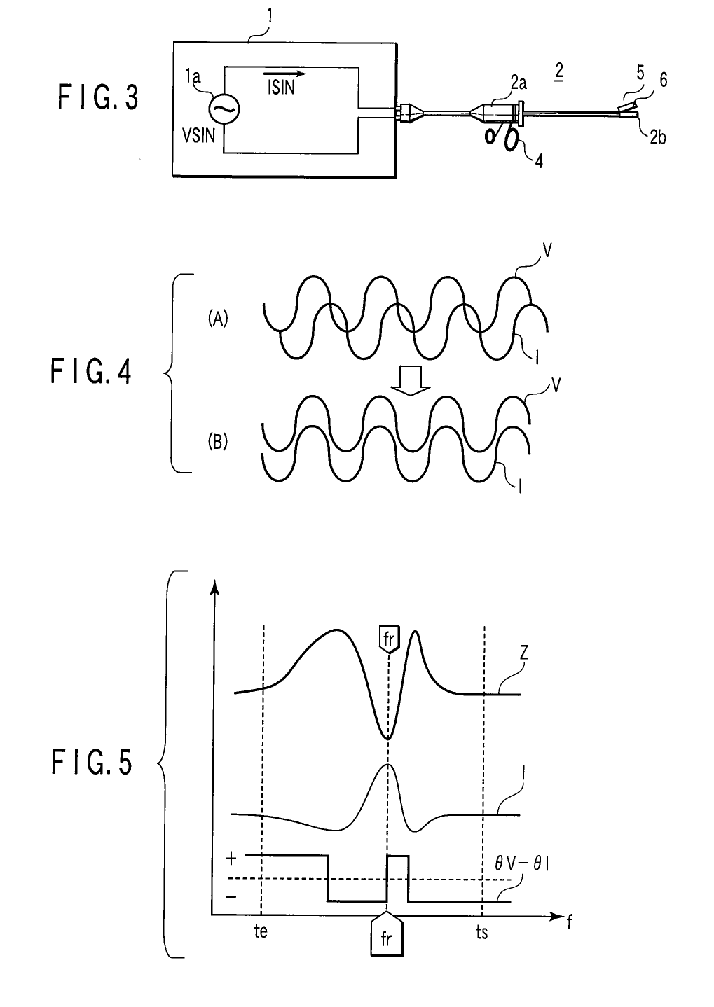

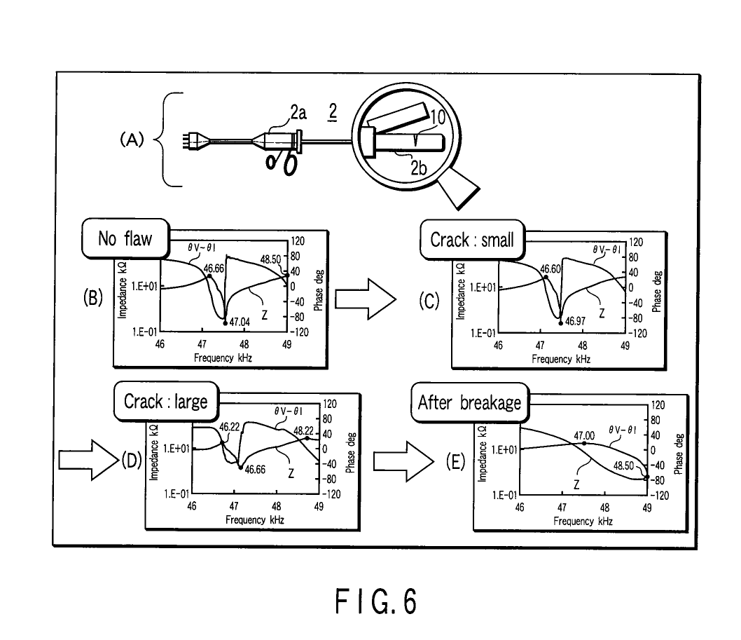

FIGS. 3 to 5 are views for explaining a method of controlling ultrasonic drive in an ultrasonic operation system. In FIG. 3, in an ultrasonic oscillator circuit 1a, a sinusoidal drive voltage VSIN is generated. When a sinusoidal drive current ISIN corresponding to the sinusoidal drive voltage VSIN flows into the ultrasonic vibrator inside the hand-piece main body section 2a, the ultrasonic vibrator converts the electric signal into mechanical vibration, and transmits the mechanical vibration to the distal end of the probe 2b. In the ultrasonic drive described above, when the ultrasonic vibration is output at a constant oscillation frequency, a phase difference occurs between the voltage V and the current I, and hence the drive efficiency lowers. Thus, a control circuit is provided in the ultrasonic power source unit 1, and the drive of the ultrasonic vibrator is performed while a resonance point at which a phase difference between the voltage V and the current I becomes 0 ((B) in FIG. 4) is searched for.

For example, in FIG. 5, the abscissa indicates the frequency f, and the ordinate indicates the impedance Z, current I, and phase difference (θV−θI). A value (θV−θI) indicates a phase difference. In this embodiment, a resonant frequency fr at which the phase difference (θV−θI) becomes 0 is detected by scanning for a point at which the impedance Z is minimized while consecutively changing the frequency. The control circuit 1c starts to perform the drive of the ultrasonic vibrator at the detected resonant frequency fr.

### FIRST EMBODIMENT

(A) to (E) in FIG. 6 are views for explaining a method of investigating an abnormality of a hand-piece 2 according to a first embodiment. (A) in FIG. 6 is a view showing a probe 2b part of the hand-piece 2 in an enlarging manner. This view schematically shows a state where the probe 2b has a crack 10. Here, the term crack does not necessarily imply a crack that can be confirmed with the naked eye, and includes a crack that does not appear externally, such as an internal crack, and a microcrack that appears at the early stage of metal fatigue. In the actual crack measurement, not only megascopic observation, but also microscopic observation using a magnifying glass, a metallurgical microscope or the like, and observation of a crack (microcrack) in the order of microns using an electron microscope are performed.

Measurement was conducted in detail so as to observe what variation occurs in the impedance Z and the phase difference (θV−θI) from the time when a normal probe is cracked to the time when the probe is broken. The results are shown below.

(B) to (E) in FIG. 6 are graphs showing frequency dependence of the impedance Z and the phase difference (θV−θI) which are under the PLL control, from a state where the probe is normal, through a state where the probe is cracked, to a state where the probe is broken. At (B) in FIG. 6, the probe is not yet damaged, and the impedance Z and the phase difference (θV−θI) which are in the normal state are shown. The frequency is varied by the PLL control centering around 46 to 49 kHz such that the phase difference (θV−θI) becomes zero degree. At (B) in FIG. 6, the phase difference (θV−θI) becomes also zero degree in the vicinity of 47.04 kHz at which the impedance Z becomes the lowest. Accordingly, it can be seen that this frequency is the resonant frequency.

At (C) in FIG. 6, a graph of the impedance Z and the phase difference (θV−θI) under the PLL control of the case where a small crack develops in the probe is shown. It is seen that the resonant frequency is changed from 47.04 kHz to 46.97 kHz. The minimum value of the impedance Z is slightly increased as compared with (B) in FIG. 6.

(D) in FIG. 6 is a graph showing frequency dependence of the impedance Z and the phase difference (θV−θI) under the PLL control in the case where the crack grows larger. The resonant frequency is largely shifted to 46.66 kHz. It can be seen that the graph of the impedance Z is also largely varied, and the minimum value is abruptly increased.

(E) in FIG. 6 is a graph showing the frequency dependence of the impedance Z and the phase difference (θV−θI) under the PLL control after the probe is broken. It is understood that each of the impedance Z and the phase difference (θV−θI) does not have anymore a resonance point at which the impedance Z or the phase difference (θV−θI) is abruptly changed, and the value of the impedance has largely varied. It is conceivable from the results, by paying attention to the value of the impedance Z of the hand-piece 2 under the PLL control, and by monitoring the variation with time of the impedance Z that a crack 10 which has developed in the probe 2b can be measured.

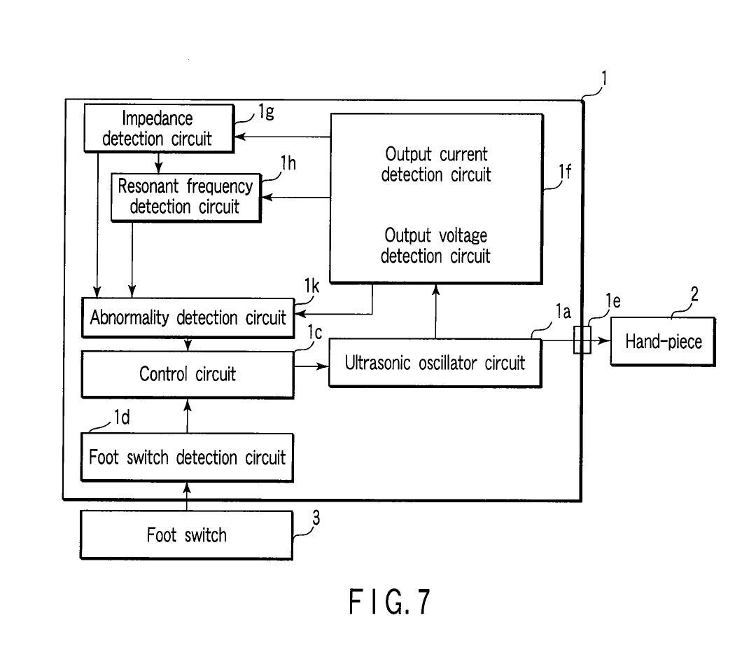

FIG. 7 is a functional block diagram for explaining a function of each unit in the ultrasonic power source unit in the ultrasonic operation system. The hand-piece 2 is connected to the ultrasonic power source unit 1 through a connector 1e. In the ultrasonic power source unit 1, an ultrasonic oscillator circuit 1a, output voltage/output current detection circuit 1f, impedance detection circuit 1g, resonant frequency detection circuit 1h, foot switch detection circuit 1d, and control circuit 1c are provided. The ultrasonic oscillator circuit 1a is a part for generating a drive signal for driving the ultrasonic vibrator inside the hand-piece 2. The foot switch detection circuit 1d is a part for detecting that the foot switch 3 has been operated by the operator.

When the foot switch 3 is operated by the operator, the operation signal is transmitted to the control circuit 1c through the foot switch detection circuit 1d. The control circuit 1c performs control such that the ultrasonic power is output from the ultrasonic oscillator circuit 1a to the hand-piece 2.

The output voltage/output current detection circuit 1f is a part for detecting an output voltage and an output current of the power supplied from the ultrasonic oscillator circuit 1a to the ultrasonic vibrator. The values of the output voltage and the output current detected by the output voltage/output current detection circuit 1f are input to the impedance detection circuit 1g and the resonant frequency detection circuit 1h. The impedance detection circuit 1g detects the impedance by using the impedance detection algorithm of the hand-piece 2 on the basis of the values of the input output voltage and the input output current, and the phase difference between them.

The resonant frequency detection circuit 1h detects a frequency actually swept at the probe 2b from the output voltage and the output current detected by the output voltage/output current detection circuit 1f and, at the same time, monitors a change in the value of the impedance transmitted from the impedance detection circuit 1g. A frequency at which the value of the impedance abruptly changes is obtained, and is detected as the resonant frequency.

The abnormality detection circuit 1k chronologically stores the value of the impedance transmitted from the impedance detection circuit 1g in the internal storage part. More specifically, the value of the impedance is saved in a memory which is the storage part at intervals of unit time of, for example, 5 msec, and the consecutively measured value of the impedance and the previously saved value of the impedance are compared with each other. Further, the value of the impedance measured at intervals of 5 msec is compared with plural values of the impedance such as values measured 5 msec ago, 10 msec ago, 15 msec ago, and so on, thereby judging whether or not the variation in the value of the impedance is normal. As a judging method, it is possible to set, for example, a first impedance variation value determined in advance with respect to a variation value of the impedance per unit time in the abnormality detection circuit 1k. The abnormality detection circuit 1k calculates a variation value of the value of the impedance transmitted from the impedance detection circuit 1g per unit time, compares the calculated variation value with the set first impedance variation value, and judges that the variation of the value of the impedance is abnormal when the calculated variation value exceeds the first impedance variation value.

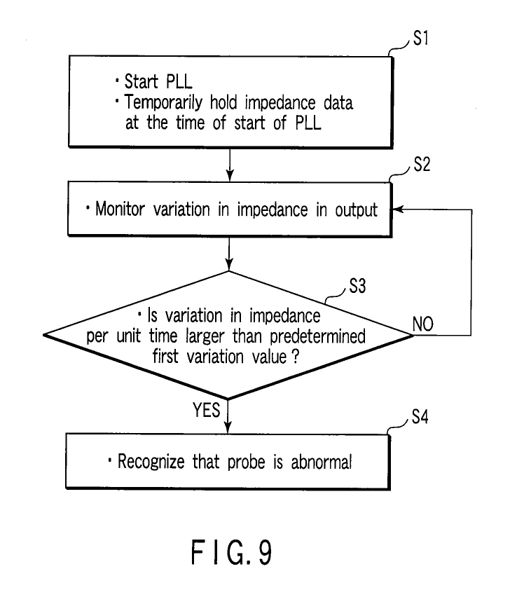

The above-mentioned flow will be described below by using the flowchart of FIG. 9. First, when an operation in an abdominal cavity of a patient is performed by using an ultrasonic probe 2b, the control circuit 1c starts the PLL control, and the abnormality detection circuit 1k detects the initial impedance of the hand-piece 2, and stores the detected value (step S1). The PLL control is the control necessary for the ultrasonic probe to perform an operation with increased energy efficiency. While the ultrasonic power is output from the ultrasonic oscillator circuit 1a to the hand-piece 2, the abnormality detection circuit 1k monitors the variation in the impedance at intervals of a fixed sampling time determined in advance (step S2). The monitored impedance value is compared with a plurality of impedance values detected previously. For example, the abnormality detection circuit 1k determines to set the sampling time at 5 msec, and compares each of 20 samples of the impedance (impedance measurement values within a period of 5 msec×20 samples)=100 msec) detected previously, or an average value of the 20 samples of the impedance detected previously with a currently detected impedance value. The abnormality detection circuit 1k compares a variation value of the impedance per unit time (100 msec) with the predetermined first impedance variation value, for example, 600Ω/100 msec (step S3), and judges that the probe is abnormal when the variation value is larger than the first impedance variation value (step S4). When the variation value is lower than the first impedance variation value, the abnormality detection circuit 1k judges that the probe 2b is normal, and returns to step S2 to continue monitoring the impedance variation.

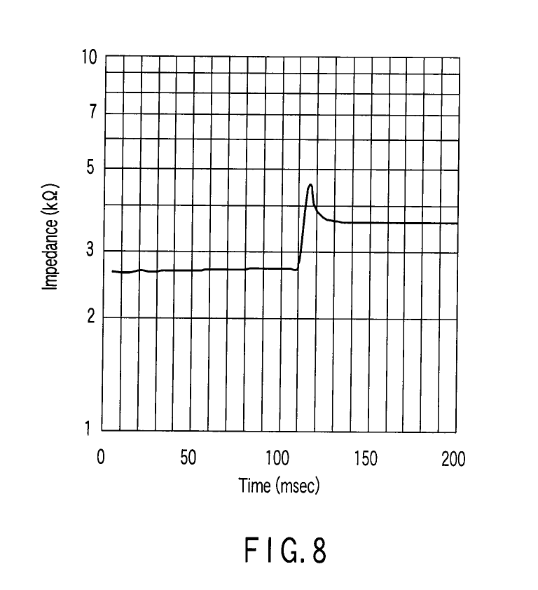

A part (corresponding to 200 msec) of the results obtained by continuously performing the measurement and by setting the sampling time at 5 msec are shown in FIG. 8 with the actually measured impedance values shown on the ordinate. It can be seen that the impedance of the hand-piece 2 varies. The impedance abruptly increases, i.e., the impedance varies from 2.65 kΩ to 4.50 kΩ between the sampling of 110 msec and sampling of 115 msec. After the impedance abruptly changes, the impedance once lowers from 4.5 kΩ to 3.6 kΩ, and thereafter remains at 3.6 kΩ. The reason why the impedance increases up to 4.5 kΩ and then decreases can be conceived that the probe in which a crack has developed is subjected to frequency rescanning by the PLL control so as to further find lower impedance, whereby the shift to a position other than the resonance point has occurred. Although the impedance is stable at 3.6 kΩ, the probe is already cracked. If the probe is further used continuously, there is the possibility of the probe being broken, and falling off in the abdominal cavity of the patient. Accordingly, the abnormality detection circuit 1k transmits a signal to the control circuit 1c to cause the control circuit 1c to stop or shut down the ultrasonic output, to thereby prevent the probe from being broken and falling off. Further, the abnormality detection circuit 1k may display a warning so as to inform the operator of the crack developing in the probe.

### (Effect)

According to this embodiment, the impedance of the hand-piece 2 is detected, the variation value of the impedance per unit time is monitored, an impedance variation value different from an impedance variation value resulting from a resection or the like of tissue by an ordinary operation is detected as an abnormality, whereby it is possible to instantaneously and easily grasp an occurrence of a crack in the probe. By virtue of the detection of the probe crack in the early stage, the medical staff can replace the probe before the breakage of the probe occurs, and safely continue the treatment of the patient.

### SECOND EMBODIMENT

A second embodiment of the present invention will be described below. Here, how to determine the first impedance variation value will be described below with reference to the data of FIG. 8. The abrupt change in the impedance occurs within several msec. When the operator performs coagulation or incision of living tissue in the abdominal cavity of the patient by an operation, the operation is performed by manipulation or grasp in units of several seconds. When the living tissue is coagulated or incised, the impedance of the probe 2b also changes by coming into contact with the living tissue. However, the variation with time is in units of seconds, and is not an abrupt change as shown in FIG. 8. Accordingly, when the first impedance variation value is to be determined, it is sufficient if the unit time is several msec to several hundred msec. In order to distinguish the impedance variation resulting from a crack in the probe, and the impedance variation resulting from contact of the probe with the living tissue from each other, the inventors have determined a number of first impedance variation values, and have repeated the experiment. As a result of this, in the case of a probe of the impedance value less than 2.65 kΩ, by setting the first impedance variation value at 2.25Ω/200 msec, the abnormality detection circuit 1k did not commit any wrong judgment. Further, in the case of a probe of the impedance value equal to 2.65 kΩ or larger, by setting the first impedance variation value at 600Ω/100 msec or 1.2 kΩ/200 msec, the abnormality detection circuit 1k did not commit any wrong judgment.

Further, as for the time at which the impedance is detected, i.e., the time at which the impedance is sampled, the instant at which a crack occurs must be accurately grasped. This is because there is the very strong possibility of a probe in which a crack is caused when an ultrasonic wave is applied thereto for a period of several hundred msec to several seconds or longer being broken and falling off, and hence it is necessary to immediately stop or shut down the ultrasonic output. As is apparent from FIG. 8, the crack of the probe 2b has occurred between 5 msec and 10 msec, and hence it is desirable that the detection interval of the impedance be 10 msec or less.

### (Effect)

As for the first impedance variation value determined in advance with respect to a variation value of the impedance per unit time, in the case of a probe of an impedance value of less than 2.65 kΩ, the first impedance variation value is set at 2.5Ω/200 msec, and in the case of a probe of an impedance value of 2.65 kΩ or larger, the first impedance variation value is set at 600Ω/100 msec or 1.2 kΩ/200 msec, whereby the abnormality detection circuit 1k did not commit any wrong judgment. By this method of setting the first impedance variation value, it is possible to accurately and easily distinguish the impedance variation of the ordinary operation and the variation in the impedance due to a crack in the probe 2b from each other.

Further, by setting the interval of sampling of the impedance at 10 msec or less, it is possible to grasp the accurate time at which the crack is caused, stop or shut down the ultrasonic output accordingly, and prevent breakage or falling off of the probe greater than the crack.

### THIRD EMBODIMENT

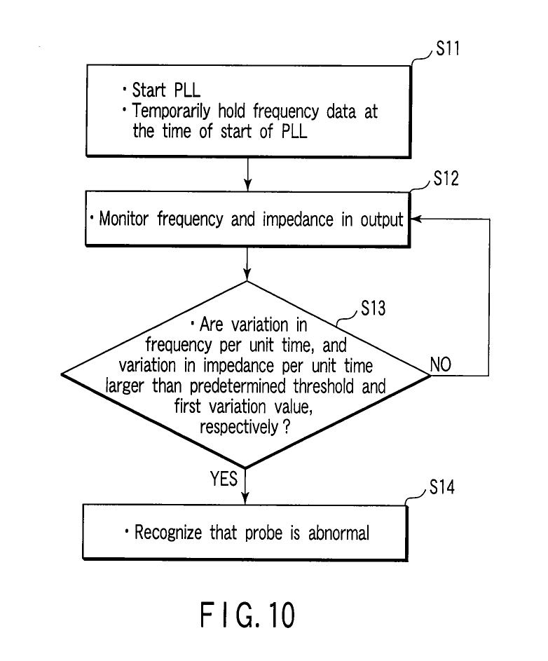

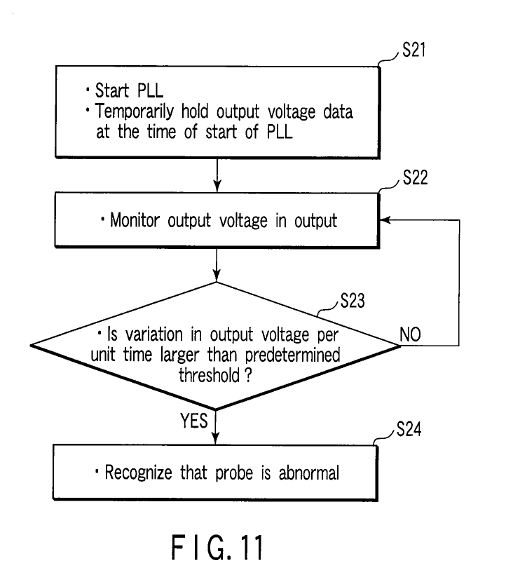

A third embodiment of the present invention will be described below with reference to the block diagram of FIG. 7 and the flowchart of FIG. 10. Here, only the parts different from the first and second embodiments will be described below. Steps S1, S2, and S3 of the flowchart of FIG. 9 correspond to steps S11, S12, and S13 of the flowchart of FIG. 10, and hence detailed description of them will be omitted.

In FIG. 7, a resonant frequency detection circuit 1h detects a resonant frequency on the basis of the output voltage and the output current from the output voltage/output current detection circuit 1f, and the variation in the impedance value from the impedance detection circuit 1g. The resonant frequency is varied by a crack in the probe 2b. This is apparent from (B) to (E) in FIG. 6. The variation in the resonant frequency per unit time is compared with a predetermined threshold. When the variation is larger than the threshold, the variation is judged to be an abnormality of the probe. Further, it is also possible, only when the variation value of the impedance shown in the first embodiment is larger than the first impedance variation value determined in advance for the impedance, to judge the variation value of the impedance to be abnormal (step S13). As described above, the judgment of the abnormality can be made only on the basis of the resonant frequency. However, by making the abnormal variation in the impedance the condition of the abnormality, a more accurate and appropriate judgment can be made.

### (Effect)

In addition to judging the impedance variation value to be abnormal, when the variation in the resonant frequency is larger than the predetermined threshold, the variation in the resonant frequency is judged to be abnormal. By judging the case where these two conditions are satisfied (both the abnormality of the impedance variation value, and the abnormality of the resonant frequency variation) to be abnormal, a more accurate and appropriate judgment can be made, and a more accurate and appropriate stoppage or shutdown of the ultrasonic output can be performed.

### FOURTH EMBODIMENT

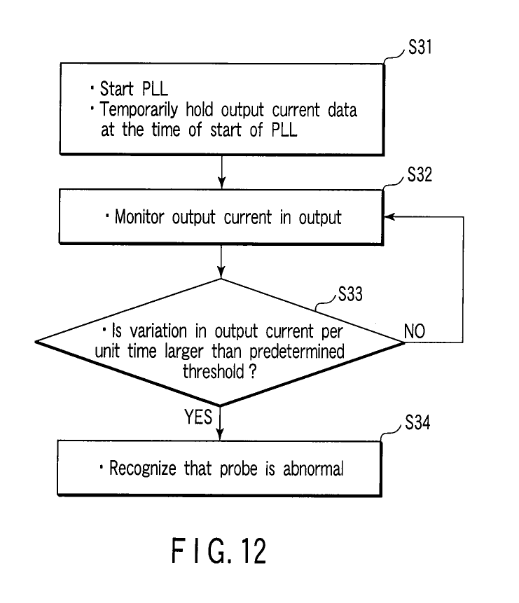

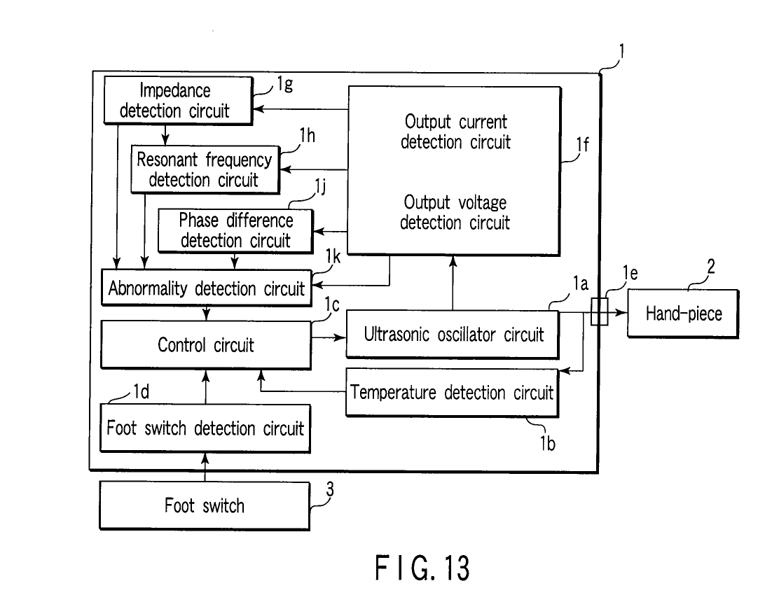

A fourth embodiment of the present invention will be described below with reference to the block diagram of FIG. 7, and the flowcharts of FIGS. 11 and 12. Here, only parts different from the first, second, and third embodiments will be described.

An output voltage/output current detection circuit 1f is a detection part for detecting an output voltage and an output current in the output, and data of these detected output voltage and the output current is input to an abnormality detection circuit 1k. In the abnormality detection circuit 1k, a first voltage variation value or a first current variation value of a variation value of the output voltage or the output current per unit time determined in advance is set. Variation values of the input output voltage and the input output current are compared with the thresholds, and when it is judged that variation values of the input output voltage and the input output current are values larger than the first voltage variation value and the first current variation value, respectively (step S23 in FIG. 11, and step S33 in FIG. 12), it is judged that the probe is abnormal (step S24 in FIG. 11, and step S34 in FIG. 12), and the ultrasonic output is stopped or shut down.

### (Effect)

The output voltage or the output current which is being output is subjected to variation due to a crack in the probe 2b. Particularly, the values of the output voltage and the output current can be measured with higher accuracy than the impedance or the frequency. Accordingly, the variation values of the output voltage or the output current is compared with the predetermined first voltage variation value or the first current variation value, and judging that the probe is abnormal on the basis of the comparison makes it possible to grasp a crack in the probe more accurately and appropriately.

### FIFTH EMBODIMENT

A fifth embodiment will be described below with reference to the block diagram of FIG. 13. This block diagram resembles the block diagram of FIG. 7, and includes a phase difference detection circuit 1j, and a temperature detection circuit 1b in addition to the constituents of the block diagram of FIG. 7. It is known that the phase difference (θV−θI) between the output voltage and the output current detected by the phase difference detection circuit 1j varies due to a crack in the probe 2b. Further, it has been found that the temperature variation of the hand-piece 2 is due to the crack of the probe 2b by measuring the temperature of the hand-piece 2. More specifically, the capacity of the hand-piece 2 is correlated with the internal temperature thereof, and hence by measuring the capacity thereof the temperature can be measured. Thus, these variation values are compared with the thresholds, and when it is judged that the variation values are values larger than the thresholds, it is judged that the probe is abnormal, and the ultrasonic output is stopped or shut down.

### (Effect)

By measuring the phase difference (θV−θI) or the temperature of the hand-piece 2, a crack in the probe can be grasped more accurately and appropriately.

### SIXTH EMBODIMENT

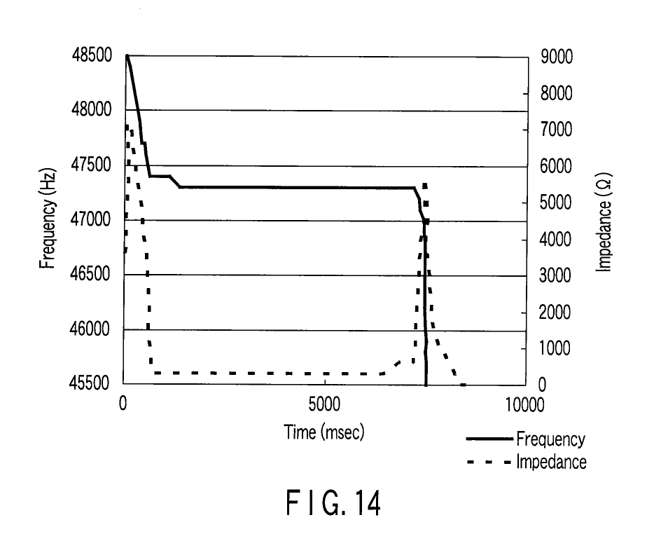

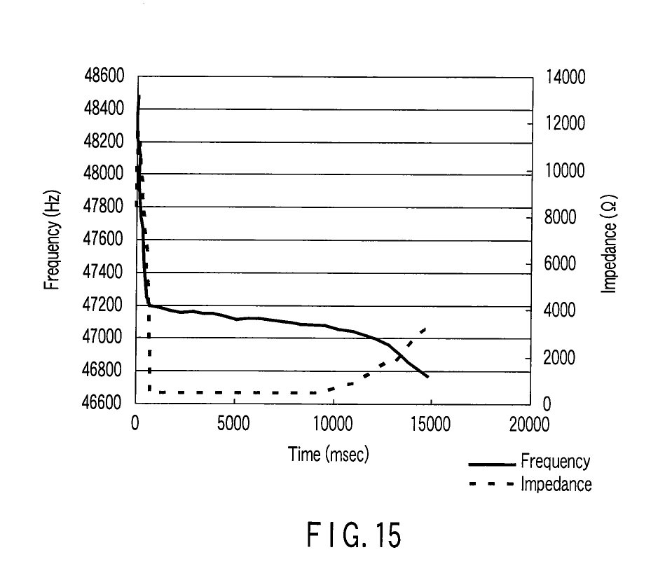

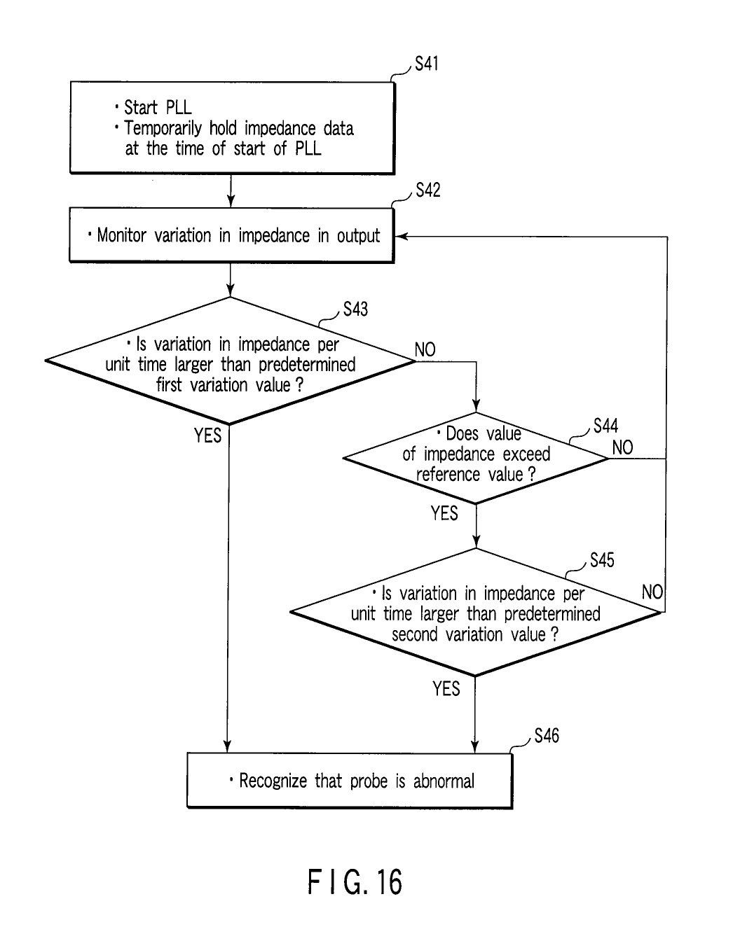

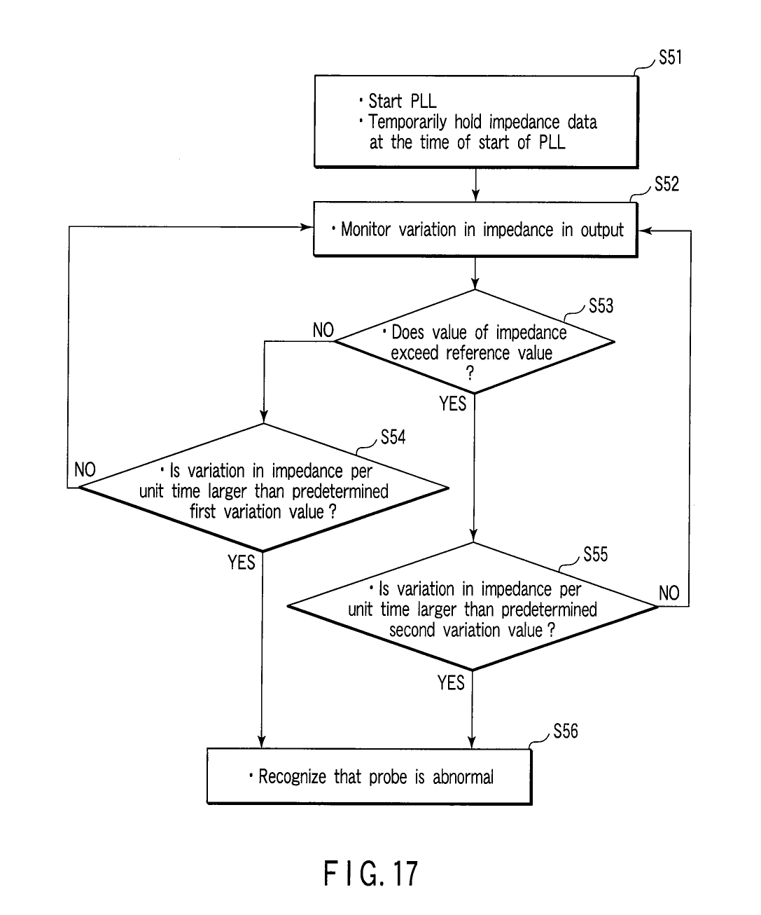

A sixth embodiment will be described below with reference to FIGS. 14 to 17. FIG. 14 is a graph showing time dependence of the frequency in addition to the time dependence of the impedance shown in FIG. 8 described in the second embodiment. As for a probe, a probe different from the probe used in the measurement of FIG. 8 is used. The variations in the frequency and impedance up to 700 msec are those at the start-up time, and do not indicate the abnormality of the probe. In the range up to 7000 msec, the frequency or the impedance is stable in the vicinity of 47.3 kHz or 300Ω. At about 7450 msec, the frequency abruptly lowers, and the impedance abruptly increases up to 5700Ω, and then abruptly lowers. It can be seen that a crack has occurred in the probe 2b at the time of the variation. By repeating the similar experiment, it has been found that a crack occurs when the graph exhibits the similar variation. However, there have been cases where a crack occurs even when the graph does not exhibit such a variation. A graph obtained in such a case is shown in FIG. 15. In FIG. 15, the variation value of the impedance does not vary so abruptly as FIG. 14. However, the value of the impedance itself increases to exceed 600Ω at 10000 msec, and exceeds 1 kΩ at 11300 msec, the value of the impedance being normally about 300Ω. The value of the impedance further continues to increase, and reaches 3.2 kΩ at the time of 15000 msec. It can be conceived that this is attributable to the crack generation mechanism. This crack is not a type of crack that abruptly extends from a locally generated crack, and the crack is considered to be of a case where fine cracks in the probe, e.g., microcracks are joined together to consequently form a large crack. Flowcharts for detecting such a variation are shown in FIGS. 16 and 17. Steps S1 and S2 in the flowchart of FIG. 9 correspond to steps S41 and S42 in the flowchart of FIG. 16, and steps 51 and 52 in the flowchart of FIG. 17, and thus detailed description of them will be omitted.

The abnormality detection circuit 1k compares the variation in the impedance per unit time (100 msec) with a predetermined first impedance variation value, for example, 600Ω/100 msec (step S43), and judges that the probe is abnormal when the variation is larger than the first impedance variation value (step S46). When the variation is smaller than the first impedance variation value, the abnormality detection circuit 1k compares the value of the impedance of the probe with a predetermined reference value (step S44), and if the impedance value does not exceed the reference value, the abnormality detection circuit 1k judges that the probe 2b is normal. Then, the abnormality detection circuit 1k returns to step S42 to continue monitoring the variation in the impedance.

Conversely, if the impedance value exceeds the reference value, the variation value of the impedance is compared with a predetermined second impedance variation value (step S45). When the variation value of the impedance is larger than the second impedance variation value, the probe is judged to be abnormal (step S46). In this case, by setting the predetermined second impedance variation value at a value lower than the predetermined first impedance variation value, it is possible to perform crack detection with higher accuracy and precision.

In the flow shown in FIG. 16, the variation value of the impedance is first compared with the first variation value. However, as in the flow shown in FIG. 17, the value of the impedance may be first compared with the predetermined reference value (step 53), when the value is equal to or smaller than the reference value, the variation value of the impedance may be compared with the predetermined first variation value (step S54), and when the variation value of the impedance exceeds the predetermined first variation value, the variation value of the impedance may be compared with the predetermined second variation value (step S55).

As the result of conducting an experiment on the above flow by using actual probes, in a certain probe, when the predetermined reference value of the impedance, the first variation value, and the second variation value are set at 1.7 kΩ, 1.5 kΩ/◯◯ msec, and 400Ω/◯◯ msec, respectively too, the abnormality detection circuit 1k did not commit any wrong judgment.

By making a judgment in accordance with the above flow, it is possible to detect not only the variation shown in FIG. 14, but also the abnormality in the probe shown in FIG. 15 without overlooking the minute variation shown in FIG. 15. When it is judged that the probe is abnormal (steps S46 and S56), the abnormality detection circuit 1k transmits, in order to prevent the probe from being broken or falling off, a signal to the control circuit 1c so as to cause the control circuit 1c to stop or shut down the ultrasonic output. Further, the abnormality detection circuit 1k may display a warning so as to inform the operator of the crack developing in the probe.

### (Effect)

According to this embodiment, the impedance of the hand-piece 2 is detected, the value of the impedance is compared with the predetermined reference value, and at the same time, the variation value of the impedance per unit time is compared with the predetermined first variation value and the second variation value, whereby it is possible to detect an impedance variation value different from an impedance variation value resulting from a resection or the like of tissue by an ordinary operation as an abnormality with high accuracy and precision, and instantaneously and easily grasp an occurrence of a crack in the probe. By virtue of the detection of the probe crack in the early stage, the medical staff can replace the probe before the breakage of the probe occurs, and safely continue the treatment of the patient.

What is claimed is:

1. A power supply apparatus for operation for outputting power to a surgical instrument comprising:
an impedance detection section for detecting the impedance of the surgical instrument from the power in the output; and
an abnormality detection section for detecting whether or not a variation value of the impedance per unit time exceeds a predetermined first impedance variation value.

2. The power supply apparatus for operation according to claim 1, wherein the abnormality detection section further detects whether or not a variation value of a resonant frequency per unit time exceeds a predetermined threshold.

3. A power supply apparatus for operation for outputting power to a surgical instrument comprising:
a detection section for detecting an output voltage or an output current from the power in the output; and
an abnormality detection section for detecting whether or not a variation value of the output voltage or the output current per unit time exceeds a predetermined first voltage variation value or a predetermined first current variation value.

4. The power supply apparatus for operation according to claim 1, wherein each of intervals at which the impedance is detected is 10 msec or less.

5. The power supply apparatus for operation according to claim 1, wherein the first impedance variation value is 600Ω/100 msec or more.

6. The power supply apparatus for operation according to claim 1, wherein the abnormality detection section stops outputting the power to the surgical instrument when the variation value of the impedance per unit time exceeds the first impedance variation value.

7. The power supply apparatus for operation according to claim 3, wherein the abnormality detection section stops outputting the power to the surgical instrument when the variation value of the output voltage or the output current exceeds the predetermined first voltage variation value or the predetermined first current variation value.

8. The power supply apparatus for operation according to claim 1 or 3, wherein the surgical instrument is provided with an ultrasonic vibrator, and a probe for transmitting the vibration of the ultrasonic vibrator to a distal end thereof, and
the output power is ultrasonic power for driving the ultrasonic vibrator.

9. The power supply apparatus for operation according to claim 1, wherein the abnormality detection section further detects whether or not the variation value of the impedance per unit time exceeds a second impedance variation value when a value of the impedance detected by the impedance detection section exceeds a predetermined reference value.

10. The power supply apparatus for operation according to claim 9, wherein the second impedance variation value is smaller than the first impedance variation value.

11. The power supply apparatus for operation according to claim 10, wherein the abnormality detection section stops supplying the power to the surgical instrument when the variation value of the impedance per unit time exceeds the first variation value, or when the value of the impedance exceeds the reference value, and the variation value of the impedance per unit time exceeds the second impedance variation value.
